(19) United States
(12) Patent Application Publication
(10) Pub. No.: US 2010/0030170 A1
(43) Pub. Date: Feb. 4, 2010

(54) Absorptive Pad

(76) Inventors: Keith Alan Keller, Spartanburg, SC (US); T. Andrew Canada, Chesnee, SC (US); D. Brian Hall, Simpsonville, SC (US); W. Mark Housley, Simpsonville, SC (US)

Correspondence Address: Legal Department (M-495), P.O. Box 1926, Spartanburg, SC, 29304, US

(21) Appl. No.: 12/496,771

(22) Filed: Jul. 2, 2009

Related U.S. Application Data
(60) Provisional application No. 61/085,592, filed on Aug. 1, 2008.

(51) Int. Cl. A61L 15/46

(52) U.S. Cl. 604/360

(57) ABSTRACT

This disclosure relates to a substrate comprising multiple layers which are designed to provide moisture absorption, non-adherence, and antimicrobial properties to a contact surface. The substrate is minimally comprised of (a) at least one skin contact layer of apertured hydrophobic polymeric film, (b) at least one layer of absorptive material that contains a reinforcement material, and (c) at least one layer of liquid impermeable film backing. The substrate may also include a layer of material that contains an antimicrobial agent. The substrate may be ideally suited for use in the medical field as an absorptive pad for end-uses such as wound care (e.g. burn care), surgery care, and incontinence care.

### CROSS REFERENCE TO RELATED APPLICATIONS

This application claims priority to U.S. Provisional Patent Application No. 61/085,592, entitled “Absorptive Pad” which was filed on Aug. 1, 2008.

### TECHNICAL FIELD

This disclosure relates to a substrate comprising multiple layers which are designed to provide moisture absorption, non-adherence, and antimicrobial properties to a contact surface. The substrate is minimally comprised of (a) at least one skin contact layer of apertured hydrophobic polymeric film, (b) at least one layer of absorptive material that contains a reinforcement material, and (c) at least one layer of liquid impermeable film backing. The substrate may also include a layer of material that contains an antimicrobial agent. The substrate may be ideally suited for use in the medical field as an absorptive pad for end-uses such as wound care (e.g. burn care), surgery care, and incontinence care.

### BACKGROUND

In the medical field, and in the area of wound care particularly, it is well-established that many factors, including the amount of moisture present at a wound site, affects how quickly a wound will heal. Generally speaking, having an excessive amount of moisture present at a wound site, especially when combined with the warm environment provided by the body, leads to undesirable growth of microbes such as bacteria and production of protease enzymes in the wound. Such growth can cause further damage to healthy cells and delay the healing process. However, insufficient moisture at the wound site can cause eschar formation and scarring and may cause the medical dressing to adhere to the wound. Absorptive materials such as gauzes, hydrogels, swellable fibers, foams, woven textiles and the like have been incorporated into medical dressings for the purpose of controlling the wound moisture content. Once the local saturation limit of the materials is reached, the materials are generally incapable of absorbing additional fluid.

Additionally, if the dressing adheres to the wound, subsequent removal of the dressing may cause undue discomfort to the patient as well as disrupt newly granulated tissue. Infection of the wound may also be compounded when a medical dressing is removed and portions of the dressing remain behind in the wound itself, particularly if the dressing is already colonized with pathogenic microbes. Thus, it is important that the dressing maintains its physical integrity when exposed to stress, such as during removal from the wound, in order to prevent additional complications and delays in healing.

There have been previous attempts by others to combine multiple layers together into a single medical dressing or device to address one or more of these problems. For example, U.S. Pat. No. 4,173,046 to Gallagher discloses an absorptive patient underpad. The underpad includes four layers laminated together—a perforated closed cell foam layer, a perforated hydrophobic plastic sheet layer, a layer of hydrophilic material, and another layer of hydrophobic plastic sheeting. The patient contact surface consists of a closed cell foam plastic material, which provides cushioning for bedridden patients. The underpad fails to provide for the delivery of an antimicrobial material for improving the rate of healing of the wound and furthermore, fails to teach a dressing that provides a one-way flow of moisture from the fluid source and into the underpad.

U.S. Pat. No. 5,478,335 to Colbert teaches an absorbent device comprising an absorbent pad covered with two layers of flexible polymeric nets (i.e. an outer cover sheet and an intermediate layer). The intermediate layer is oriented such that strike back of fluid (movement of moisture back toward the wound) to the patient is minimized. The outer surface of the device facing away from the user may also include a barrier film having adhesive strips located thereon, in order to adhere the device to a garment. The inclusion of multiple layers of polymeric net in this device results in a higher cost dressing.

U.S. Pat. No. 4,667,665 to Blanco et al. discloses the Exu-Dry™ product, a multi-layer wound dressing, available from Smith and Nephew. The dressing includes a first layer of high density polyethylene, inner layers of highly absorbent cellulose and rayon/polyester blended materials, an anti-shear layer of perforated high density polyethylene, and a wound contact layer of perforated high density polyethylene. The inclusion of multiple layers of perforated film in this dressing results in a higher cost dressing. Additionally, the dressing fails to provide for the delivery of an antimicrobial material for improving the rate of healing of the wound, and furthermore, fails to teach a dressing that provides a one-way flow of moisture from the wound and into the dressing.

U.S. Pat. No. 4,948,651 to DeBusk et al. describes a multi-layer burn sheet. The layers of the burn sheet, which are laminated together, include a liquid permeable, perforated plastic web; an absorbent cellulose layer; a scrim layer; and a liquid impermeable plastic web. The scrim layer is comprised of small, multifilament and calendared yarns and is included to impart strength to the burn sheet so that it may also be utilized as a patient transfer sheet. The patent contact surface of the burn sheet consists of the liquid permeable, perforated plastic web layer. This burn sheet fails to provide for the delivery of an antimicrobial material for improving the rate of healing of a burn and furthermore, fails to teach a burn sheet that provides a one-way flow of moisture from the wound and into the burn sheet which provides optimum conditions for healing. The absorptive pad of the present invention includes the desired features of non-adherence to a wound, control of moisture flow away from the wound and preventing it from transferring back toward the wound, and the optional inclusion of an antimicrobial agent. The absorptive pad is conformable to the wound and comfortable for the patient. Furthermore, it minimizes disruption of new cell growth in and around the wound and does not cause irritation of the wound. Thus, the present disclosure addresses and overcomes the problems described above. For these reasons and others that will be described herein, the present absorptive pad represents a useful advance over the prior art.

### SUMMARY

Provided herein is an absorptive pad comprising the sequential layers of at least one skin-contact layer of apertured hydrophobic film; at least one layer of absorptive material that contains a reinforcement material; optionally, at least one layer of absorptive material; optionally, at least one layer of non-electrically conductive absorptive material that contains an antimicrobial agent; and at least one layer of occlusive film; wherein each of the layers are arranged substantially coextensive with each other.

Also provided herein is an absorptive pad comprising the sequential layers of at least one skin-contact layer of apertured hydrophobic film; at least one layer of absorptive material that contains a reinforcement material; at least one layer of absorptive material; optionally, at least one layer of non-electrically conductive absorptive material that contains an antimicrobial agent; and at least one layer of occlusive film; wherein each of the layers are arranged substantially coextensive with each other.

Also provided herein is an absorptive pad comprising the sequential layers of at least one skin-contact layer of apertured hydrophobic film; at least one layer of absorptive material that contains a reinforcement material; at least one layer of absorptive material; at least one layer of non-electrically conductive absorptive material that contains an antimicrobial agent; and at least one layer of occlusive film; wherein each of the layers are arranged substantially coextensive with each other.

### BRIEF DESCRIPTION OF DRAWINGS

FIG. 1 is a schematic representation of a four-layer absorptive pad.

FIG. 2 is a schematic representation of a five-layer absorptive pad.

FIG. 3 is a schematic representation of stitch bonding in one layer of absorptive material.

FIG. 4 is a bar graph illustrating the moisture absorption of Examples 1-5.

FIG. 5A is a line graph illustrating the mechanical strength and modulus of Example 1.

FIG. 5B is a line graph illustrating the mechanical strength and modulus of the nonwoven layers of Example 1.

### DETAILED DESCRIPTION

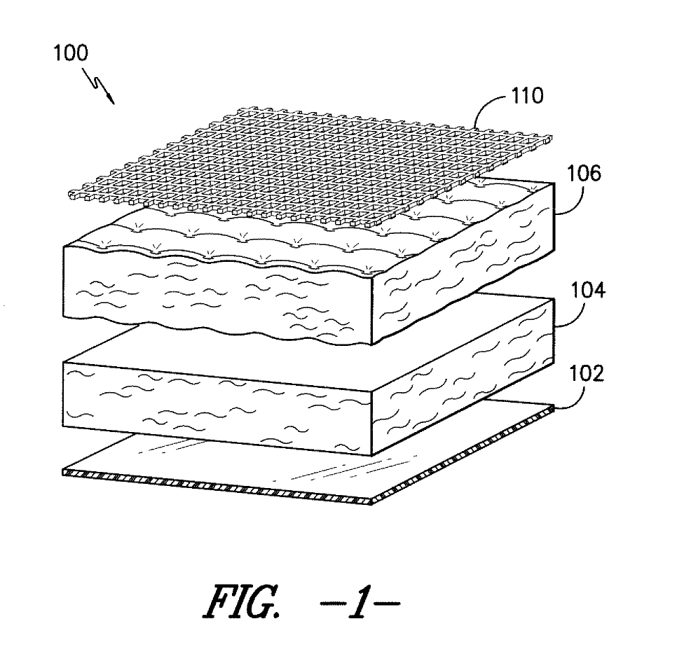

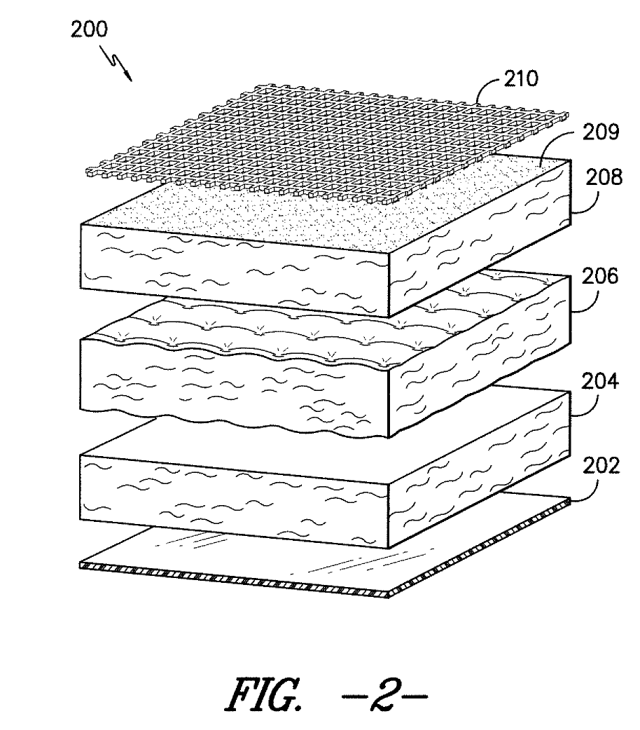

FIG. 1 and FIG. 2 are representative of the various polymeric films and textile substrates useful for making the absorptive pad in accordance with the teachings described herein. In each Figure, the bottom layer represents the layer of the absorptive pad which is located the furthest away from the intended contact surface (i.e. the outermost layer), while the top layer represents the layer of the absorptive pad which is intended to come in contact with a surface (e.g. the wound surface).

FIG. 1 illustrates a four-layer absorptive pad 100, in which the outermost layer is an occlusive polymer film layer 102. As used herein, the term “occlusive” is intended to mean impervious to fluid transmission (e.g. wound exudate) and may or may not permit gas transfer (e.g. air, water vapor, etc.).

The polymer film layer 102 may be printed on either side, although, for many applications, reverse printing on the wound-facing side of the polymer film layer 102 may be preferable. Printing may be included to provide instructions for use, such as indicating which side of the absorptive pad should contact the patient. The next layer in the multi-layer absorptive pad 100 is an absorptive layer 104 that exhibits high absorptive properties and acts as a fluid reservoir. The next layer in the absorptive pad 100 is an absorptive layer 106 that exhibits high wicking properties. It is preferable that absorptive layer 104 exhibits higher absorptivity than absorptive layer 106, so that as moisture is pulled away from the fluid source (e.g. a wound), it is prevented from moving back toward the source. The effect created by such an arrangement of absorptive layers is a one-way movement of moisture away from the fluid source.

An apertured polymeric film layer 110 forms the innermost layer of the absorptive pad 100. The apertured polymeric film layer 110 is the skin contact surface, or wound contact surface, of the absorptive pad and allows for the movement of fluid from the wound and into the absorptive pad. The apertured film layer 110 may be printed on either side, although for many applications, reverse printing on the side away from the wound may be preferable.

Polyolefin films are well-suited for layers 102 and 110, although other polymers (such as polyester or nylon) may be used. Film layers 102, 110 preferably have a thickness in the range of about 2 mils, but other thicknesses may be used. Apertured polymeric film 110 may include openings in the film of any size or shape, so long as fluid is able to move efficiently across the wound and absorptive pad interface. The openings of the film also should be of the size and spacing to provide non-adherence of the absorptive pad to the wound. For example, the apertured polymeric film 110 may have openings in the shape of slits, circles, hexagons, triangles, and the like. One example of a suitable apertured film is Delnet® apertured film, available from DelStar Technologies, Inc.

Absorptive reservoir layer 104 and absorptive layer 106 may be combined, stacked, or layered in any configuration needed for the desired end-use application of absorptive pad 100. Absorptive reservoir layer 104 is comprised of any type of textile substrate capable of providing absorbent properties to the absorptive pad 100. Absorptive layer 106 is comprised of any type of textile substrate capable of providing high wicking properties to the absorptive pad 100. Layers 104 and 106 may be independently comprised of textile substrates, such as fabrics, having a woven, nonwoven, or knit construction. Fiber types comprising the textile substrate include synthetic fibers, natural fibers, and mixtures thereof. Synthetic fibers include, for example, polyester, acrylic, polyamide, polyolefin, polyaramid, polyurethane, regenerated cellulose (i.e., rayon), and blends thereof. The term “polyamide” is intended to describe any long-chain polymer having recurring amide groups as an integral part of the polymer chain. Examples of polyamides include nylon 6; nylon 6,6; nylon 1,1; and nylon 6,10. The term “polyester” is intended to describe any long-chain polymer having recurring ester groups. Examples of polyesters include aromatic polyesters, such as polyethylene terephthalate (PET), polybutylene terephthalate (PBT), polytrimethylene terephthalate (PTT), and polytriphenylene terephthalate, and aliphatic polyesters, such as polylactic acid (PLA). “Polyolefin” includes, for example, polypropylene, polyethylene, and combinations thereof. “Polyaramid” includes, for example, poly-p-phenyleneteraphthalamid (i.e., Kevlar®), poly-m-phenyleneteraphthalamid (i.e., Nomex®), and combinations thereof. Natural fibers include, for example, wool, cotton, flax, and blends thereof.

The textile substrate may be formed from fibers or yarns of any size, including microdenier fibers and yarns (fibers or yarns having less than one denier per filament). The fibers or yarns may have deniers that range from less than about 1 denier per filament to about 2000 denier per filament or more preferably, from less than about 1 denier per filament to about 500 denier per filament, or even more preferably, from less than about 1 denier per filament to about 300 denier per filament.

Furthermore, the textile substrate may be partially or wholly comprised of multi-component or bi-component fibers or yarns, which may be splittable, or which have been partially or fully split, along their length by chemical or mechanical action. The fabric may be comprised of fibers such as staple fiber, filament fiber, spun fiber, or combinations thereof.

The textile substrate may optionally be colored by a variety of dyeing techniques, such as high temperature jet dyeing with disperse dyes, vat dyeing, thermosol dyeing, pad dyeing, transfer printing, screen printing, or any other technique that is common in the art for comparable textile products.

As one example, absorptive layer 106 is a nonwoven substrate consisting of 82% polyester staple fiber and 18% splittable nylon staple fiber, which has been stitch bonded for mechanical integrity. Absorptive reservoir layer 104 may be the same nonwoven substrate consisting of 82% polyester fiber and 18% splittable nylon fiber as described above for layer 106, except that layer 104 does not contain any stitch bonding. The presence of nylon in the absorptive layer may provide improved wickability properties to the layer when compared with a layer that is comprised of, for example, 100% polyester fiber.

The stitch bonding appears to have the effect of reducing the loft in some areas of absorptive layer 106. By not including stitch bonding in absorptive reservoir layer 104, this layer maintains a higher loft. It is believed that this arrangement of textile substrate constructions may provide for the increased absorption of layer 104 over layer 106. Furthermore, the presence of stitch bonding in layer 106 may also lead to improved in-plane wicking of fluids away from a fluid source by providing a path for the fluids to travel. Also, stitch bonding may provide channels, or holes, which aid in the movement of fluid away from the fluid source and into the absorptive pad 100. Channels, or holes, may be created by other methods in addition to, or as an alternative to, stitch bonding. For instance, needle punching techniques may be utilized to create desirable channels for fluid movement. The resulting effect provided by the presence of stitch bonding and/or the channels is a predominantly one-way flow of fluid away from the wound (or other fluid source) and into absorptive pad 100. Additional details about the configuration of stitch bonded fabrics which may be used as one or more layers of the absorptive pad of the present invention may be found in co-pending and commonly-assigned U.S. patent application Ser. No. 11/703,378 filed Feb. 7, 2007, which is entirely incorporated by reference herein.

Several configurations of the absorbent pad shown in FIG. 1 may be constructed from the layers described therein. In one instance, the absorptive pad may be comprised of one or more layers of occlusive polymer film layer 102, one or more layers of absorptive layer 104, and one or more layers of apertured film layer 110. In another embodiment, the absorptive pad may be comprised of one or more layers of occlusive polymer film layer 102, one or more layers of absorptive layer 106 and one or more layers of apertured film layer 110. In yet another instance, the absorptive pad may be comprised of one or more layers of occlusive polymer film layer 102, one or more layers of absorptive layer 104, one or more layers of absorptive layer 106, and one or more layers of apertured film layer 110.

An optional feature of the absorptive pad of the present invention is that it may contain a topical coating of an antimicrobial agent, such as silver. It is known that placing surface-available silver in contact with a wound allows the silver to become absorbed by undesirable bacteria and fungi that grow and prosper in the warm, moist environment of the wound site. Once absorbed, the silver ions kill microbes, resulting in treatment of infected wounds or the prevention of infection in at-risk wounds.

FIG. 2 illustrates a five-layer antimicrobial absorptive pad 200, in which the outermost layer is an occlusive polymer film layer 202. As before, the polymer film layer 202 may be printed on either side. The next layer in the absorptive pad 200 is an absorptive reservoir layer 204. The next layer in the absorptive pad 200 is an absorptive layer 206. Antimicrobial layer 208 is the next layer, located between absorptive layer 206 and an apertured polymeric film 210. Antimicrobial layer 208 is a textile substrate that contains an antimicrobial agent. The textile substrate of antimicrobial layer 208 may be comprised of any suitable material as described herein for absorptive layers 104 and 106. This includes various combinations of possible fiber types and fabric constructions. Absorptive reservoir layer 204 and absorptive layer 206 may be combined, stacked, or layered in any configuration needed for the desired end-use application of absorptive pad 200.

In one example, a jersey knit fabric comprised of predominantly polyester fiber on one side and nylon fiber on the opposite side may be utilized. The jersey knit fabric is arranged such that the polyester side of the fabric contacts the apertured film layer 210, and the nylon side of the fabric contacts absorptive layer 206. It may be generally known to those skilled in the art that a knit polyester fabric tends to be hydrophobic, slow to absorb liquids, and generally exhibits little or no wicking of moisture. Since polyester is hydrophobic in nature, conventional wisdom would lead one to choose a hydrophilic natural fiber, such as cotton, or a hydrophilic synthetic fiber, such as nylon, as the wound contacting side of the absorptive pad. However, it was unexpectedly discovered that by placing a hydrophobic polyester containing surface against the wound site and a hydrophilic nylon containing surface away from the wound site, a unique one-way, directional flow of fluid away from the wound site was achieved.

The antimicrobial agent comprises at least one silver-ion containing compound selected from the group consisting of silver ion exchange materials (e.g. silver zirconium phosphates, silver calcium phosphates and silver zeolites), silver particles (e.g. silver metal, nanosilver, colloidal silver), silver salts (e.g. AgCl, Ag₂CO₃), silver glass, and mixtures thereof. One preferred silver ion-containing compound is an antimicrobial silver sodium hydrogen zirconium phosphate compound available from Milliken & Company of Spartanburg, S.C., sold under the tradename AlphaSan® silver antimicrobial. Other potentially preferred silver-containing antimicrobials suitable for use herein—including silver zeolites, such as a silver ion-loaded zeolite available from Sinanen Co., Ltd. of Tokyo, Japan under the tradename Zeomic®, and silver glass, such as those available from Ishizuka Glass Co., Ltd. of Japan under the tradename Lonpure®—may be utilized either in addition to, or as a substitute for, the preferred species listed above. Various combinations of these silver-containing materials may also be utilized. In one embodiment of absorptive pad 200, antimicrobial layer 208 contains a silver-ion containing antimicrobial agent 209 which has been topically applied to the wound-facing surface of antimicrobial layer 208 (i.e. the surface of antimicrobial layer 208 that directly contacts apertured film layer 210).

Total add-on levels of silver to the target substrate may be in the range of 5 ppm to 20,000 ppm, more preferably 20 ppm to 20,000 ppm, and even more preferably 200 ppm to 20,000 ppm. Although these ranges are provided, an upper boundary limit of silver add-on levels to the target substrate may be limited only by consideration of the manufacturing economics of the product and by the potential to irritate a sensitive wound site, such that one would want to avoid excessive silver levels.

Since silver-ion containing antimicrobial agents may be added to one or more layers of the absorptive pad, the amount of silver added should be such that each silver-containing layer of the absorptive pad is preferably non-electrically conductive. “Non-electrically conductive” is defined as having a resistance in ohms per square inch of fabric of greater than about 10,000 ohms, preferably greater than about 100,000 ohms and most preferably greater than about 1×10⁹ ohms, when measured in accordance with AATCC Test Method 76-1978.

The antimicrobial agent applied to antimicrobial layer 208 may comprise non-silver compounds. These include, for example, compounds that contain copper, zinc, iodine, triclosan, polyhexamethylene biguanide (PHMB), N-halamines, chlorhexidine, quaternary ammonium complexes, and mixtures thereof, as well as common antibiotic pharmaceutical compounds. It is also contemplated that non-silver ion containing compounds may be combined with silver-ion containing compounds to form the antimicrobial agent.

Generally, the antimicrobial agent is added to the substrate in an amount from about 0.01% to about 60% by total weight of the particular finish composition; more preferably, from about 0.05% to about 40%; and most preferably, from about 0.1% to about 30%. The antimicrobial finish may include other components such as binder materials, wetting agents, odor absorbing agents, leveling agents, adherents, thickeners, and the like. The antimicrobial agent may be incorporated on and/or within absorptive layers 104 and/or 106, as well as on and/or within absorptive layers 204 and/or 206.

The inclusion of a binder material with the antimicrobial agent has been found useful in preventing the antimicrobial agent from flaking onto and/or into the wound that is being treated. Preferably, this component is a polyurethane-based binder material, although a wide variety of cationic, anionic, and non-ionic binders may also be used, either alone or in combination. Specific examples include nonionic permanent press binders (e.g., cross-linked adhesion promotion compounds, including, without limitation, cross-linked imidazolidinones available from Sequa under the tradename Permafresh®) or slightly anionic binders (including, without limitation, acrylics such as Rhoplex® TR3082 from Rohm & Haas). Other nonionics and slightly anionics are also suitable, including melamine formaldehyde, melamine urea, ethoxylated polyesters (such as Lubril QCX™, available from Rhodia), and the like. Preferably, the binder material is biocompatible such that it does not cause negative reactions in the wound. In essence, the binder materials assist in adhering the antimicrobial agent to the surface of the target substrate, such as fibers or fabrics, without negatively affecting the release of silver ions to the wound.

One exemplary acceptable method of providing an antimicrobial silver-treated fabric surface includes the application of a silver ion-containing compound and polyurethane-based binder resin from a bath mixture. This mixture of antimicrobial compound and binder resin may be applied through any technique as is known in the art, including spraying, dipping, padding, foaming, printing, and the like. By using one or more of these application techniques, a fabric may be treated with the antimicrobial compound and binder resin on only one side of the fabric, or it may be treated on both sides of the fabric. Methods of topically applying a silver-based antimicrobial finish to textile substrates are described, for example, in commonly assigned U.S. Pat. Nos. 6,584,668; 6,821,936; and 6,946,433 and in commonly assigned U.S. patent application Ser. Nos. 09/586,081; 09/589,179; 10/307,027; and 10/306,968. All of these patents and patent applications are hereby incorporated by reference.

Several configurations of the absorbent pad 200 shown in FIG. 2 may be constructed from the layers described therein. In one instance, the absorptive pad may be comprised of one or more layers of occlusive polymer film layer 202, one or more layers of absorptive layer 204, one or more layers of antimicrobial layer 208, and one or more layers of apertured film layer 210. In another embodiment, the absorptive pad may be comprised of one or more layers of occlusive polymer film layer 202, one or more layers of absorptive layer 204, one or more layers of absorptive layer 206, one or more layers of antimicrobial layer 208, and one or more layers of apertured film layer 210. In yet another instance, the absorptive pad may be comprised of one or more layers of occlusive polymer film layer 202, one or more layers of absorptive layer 206, one or more layers of antimicrobial layer 208, and one or more layers of apertured film layer 210.

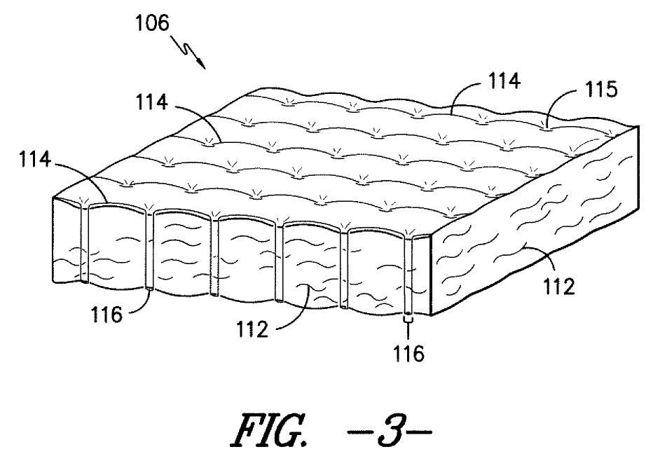

FIG. 3 is provided to illustrate the effect that stitch bonding has on absorptive layer 106 (and similarly absorptive layer 206). Stitch bonding is a bonding technique for nonwoven materials in which the fibers are connected by stitches sewn or knitted through the nonwoven web. A description of stitch bonding nonwoven webs is provided by U.S. patent application Ser. No. 11/703,376, which is incorporated by reference herein in its entirety.

The stitch bonding effect is believed to contribute to the desirable one-way flow of moisture away from the wound and into the absorptive pad, as described previously herein. As shown in FIG. 3, by adding a stitch bonding stitch 114 to absorptive layer 106, an area of decreased loft 116 surrounding the stitch 114 is created. Thus, the space between the fibers 112 surrounding the stitch is reduced. This area of decreased loft 116 is believed to result in layer 106 having a reduced ability to absorb and hold fluid, when compared to absorbent layer 104 (and similarly layer 204), which has not been stitch bonded. Stitch bonded layer 106 is arranged in direct contact with absorptive layer 104, which has not been stitch bonded. As a result, absorptive layer 104 exhibits increased absorbency properties, when compared with the stitch bonded layer 106. Thus, the sequential arrangement of layers 106 and 104, which are in direct contact with one another, allows moisture absorbed from the wound to pass first to stitch bonded absorptive layer 106 and then to non-stitch bonded absorptive layer 104.

One effect of the stitch bonding is to enhance the in-plane wicking of fluids away from a localized fluid source. The presence of stitches 114 increases the contact area of any moisture with other layers within the absorptive pad. Additionally, the presence of the holes (or channels) 115 created by the stitch bonding, or by other techniques such as needle punching, provides channels for passage of fluids away from the fluid source. The result of stitch bonding is a one-way movement of fluid away from the fluid source (e.g. a wound) and into the absorptive pad 100 (and similarly absorptive pad 200). The absorptive layer 106 may be stitch bonded with any natural or synthetic fiber type. In one embodiment, a continuous polyester fiber is employed as the stitch bonding fiber.

Additionally, absorptive layer 106 exhibits increased strength due to the stitches 114 provided throughout the layer 106. Thus, the presence of multiple stitches 114 provides reinforcement to absorptive layer 106. It is believed that the inclusion of reinforced, absorptive layer 106 provides an overall increase in strength to absorptive pad 100 (and similarly absorptive pad 200). While the presence of stitch bonding stitches 114 provides reinforcement material for layer 106 in this embodiment, it is contemplated that reinforcement material for absorptive layer 106 may be provided by other means. For example, reinforcement materials may include, or may be represented by: the presence of patterned adhesive, by linear ultrasonic fusion, by the use of long staple fiber utilized in a particular fabric construction (e.g. staple fiber length >1.5 inches), by the inclusion of a scrim layer in a nonwoven fabric, by patterned thermal fusion, by sewing of the edges of a textile substrate, and the like, and by any combination of these methods. Thus, it may be preferable that absorptive layer 104 (and similarly absorptive layer 204) is free from stitch bonding and from any other reinforcement material. However, it is contemplated herein that any of the film layers, either the apertured film layer or the occlusive film layer, may be reinforced by any means disclosed herein for providing reinforcement to the absorptive pad.

The combination of stitch bonding and the orientation of the textile substrate in the absorptive pad may be optimized to provide greater uniformity of strength in the length and width direction of the absorptive pad. Furthermore, stitch bonded fabrics may be incorporated into the absorptive pad in any orientation desired to provide the necessary physical properties to the end-use product.

The production process for making the absorptive pad may include bringing together the various layers of the pad, such as from rolled goods; cutting the layers into the desired shape; and combining the layers together for use, such as by sewing or thermal sealing of the edges, to yield a finished, properly dimensionalized product. Alternatively, the layers could be laminated together at wide widths, as the layers are taken from the rolled goods, and then the laminated layers may be cut into the properly dimensionalized product. It is contemplated that each finished product will be individually packaged in standard medical packaging material and then sterilized, such as by gamma irradiation.

Each of the layers comprising the absorptive pad of the present invention is arranged substantially coextensive with each another. The layers of the absorptive pad may be joined together by any conventional process. For example, the layers may be joined by using adhesives, by sewing the layers together, via thermal sealing of the edges, by spot lamination, by ultrasonic lamination, and the like, and combinations thereof.

The absorptive pad described herein may be of any shape or size as needed to treat a particular wound or medical condition. For instance, the absorptive pad may be provided as square pads, or they may be provided in the shape of a garment which can be worn by a patient, such as a vest.

Although reference has been made throughout this description to the absorptive pad as being intended for use to treat wounds, it should be readily apparent to those of skill in the art that the absorptive pad described herein may be suitable for use in treating various types of wounds or medical conditions wherein moisture absorbency and/or antimicrobial properties are desirable. Non-limiting examples of suitable intended applications include the use of the absorptive pad as a burn pad, a patient transfer sheet, an incontinence pad, and the like.

The absorptive pad may also be utilized as a drug delivery apparatus by incorporating certain compounds into one or more layers of the absorptive pad which may be released for use in or on a patient. For instance, these compounds may include antibiotics, pain relievers, peptides, growth factors, anti-inflammatory agents, enzymatic or other debriding agents, and the like, and mixtures thereof.

Other additives may be present on and/or within the fabric or yarn comprising the absorptive pad, including antistatic agents, optical brightening compounds, opacifiers (such as titanium dioxide), nucleating agents, antioxidants, UV stabilizers, fillers, permanent press finishes, softeners, lubricants, curing accelerators, adhesives, and the like. The absorptive layers may also be coated or printed or otherwise aesthetically modified. Printing may be achieved, for example, by screenprinting or flexographic printing techniques.

The absorptive pad itself may further include additional additives as needed for desirable end-use attributes. One exemplary additive includes superabsorbing polymers such as polyacrylic acid, polyacrylamide-containing polymers, polyvinyl alcohol, and the like, and mixtures thereof.

The absorptive pad may be of any thickness, depending on the construction of the fabric and the desired level of padding and/or absorbency needed. It may, however, be preferred that the thickness of the absorptive pad is between about 0.0625 inches and about 2 inches, more preferably between about 0.125 inches and about 0.5 inches.

An additional advantageous feature of the absorptive pad of the present invention is its ability to substantially maintain its original color, despite the optional presence of effective amounts of a silver-ion containing antimicrobial agent. The elimination of color normally associated with the inclusion of silver-based antimicrobials is highly beneficial and desirable. The absorptive pad (preferably, white-colored) allows users thereof and their health care providers to monitor the exudates from a wound. Further, the absorptive pad generally exhibits long-term color stability (that is, the color does not change significantly over time while in production, transit, or storage). Finally, because the absorptive pad is not discolored by the addition of the silver-ion containing antimicrobial agent, a variety of substrate colors may be utilized. Colored substrates may be achieved by dyeing or coloring to any desired shade or hue with any type of colorant, such as, for example, pigments, dyes, tints, polymeric colorants, and the like.

The following examples further illustrate the present absorptive pad but are not to be construed as limiting the invention as defined in the claims appended hereto. All parts and percents given in these examples are by weight unless otherwise indicated.

### Sample Creation and Evaluation

### A. Substrate Descriptions

Example 1 was comprised of the following sequential layers
(a) an apertured polyethylene film layer (Delnet® AC530WHT-E) as the skin contact layer;
(b) a 4.8 oz/yd² needlepunched nonwoven layer formed from a 65% by weight of a 2.25 denier, 4.0 inch polyester staple fiber and 35% by weight of a 6 denier, 16 segment splittable fiber having a 46:54 weight ratio of nylon to polyester. which has been stitch bonded with a continuous filament of 1 ply 150 denier 34 filament polyester yarn in the machine direction at 10.7 courses per inch (needles per inch of fabric in the transverse direction) and 10.2 wales per inch (needle stitches per inch of fabric in the machine direction);
(c) the same 4.8 oz/yd² needlepunched nonwoven layer as described above in (b), except without the stitch bonding; and
(d) an occlusive film layer comprised of 1.7 mil embossed taffeta polyethylene film as the outermost layer.

All of the layers (a)-(d) were combined together and cut into 24 inch by 36 inch samples. The layers were sewn together using conventional sewing techniques. Example 1 is illustrated by FIG. 1.

Example 1 was evaluated for moisture absorption, moisture wicking, and strength, according to the test procedures described herein.

Example 1A was comprised of the same layers as Example 1. However, Example 1A was not sewn at the edges and the sample size was approximately 4″ by 14″.

Each of the layers comprising Example 1 was tested separately for moisture absorbance and some were tested for strength:
Example 2 was the Delnet® AC530WHT-E apertured polyethylene film.
Example 3 was the stitch bonded nonwoven layer described as “c” above.
Example 4 was the non-stitch bonded nonwoven layer described as “b” above.
Example 5 was the polyethylene occlusive film described as “d” above.

Example 6 is the same as Example 1, except no stitch bonding is present in the layers and the sample size approximately 4″×14″.

Example 7 is the same as Example 1, except that both nonwoven layers are stitch bonded and the sample size is approximately 4″×14″.

Example 8 is the same as Example 1, except that an antimicrobial layer was added to the absorptive pad of Example 1. Example 8 is illustrated by FIG. 2.

For Example 8, the antimicrobial layer was a double jersey knit (circular knit), multi-polymer fabric sold by Milliken & Company. The fabric was single layer of fabric comprised of approximately 66% continuous filament polyamide yarn, 19% continuous filament polyester yarn, and 15% continuous filament spandex yarn. The polyamide yarn was comprised of 2 plies of 40 denier/34 filament count nylon 6 fiber that was exposed to a texturing process prior to knitting. The polyester yarn was comprised of single ply 70 denier/34 filament count fiber that was exposed to a texturing process prior to knitting. The spandex yarn was comprised of 55 denier/3 filament count fiber.

The fabric was knitted in such as manner as to give a distinct nylon side and a distinct polyester side. The polyester side of the fabric was exposed to a face-finishing process known as sanding.

The fabric was passed through a bath containing an antimicrobial formulation (further described below) and subsequently through squeeze rollers to achieve a wet pick-up of about 85%. The fabric was then dried in a tenter frame to remove excess liquid.

A solution was prepared according to the formulation shown below and was applied to the antimicrobial layer, i.e. the jersey knit fabric.

Formulation

| Component | Amount (% w/w) |
|---|---|
| Water | 67.8 |
| Witcobond ® UCX-281F (polyurethane binder) | 14.2 |
| AlphaSan ® RC 2000 (antimicrobial agent, 10% Ag) | 18.0 |

Comparative Example 1 was a commercially available multi-layered dressing known by the tradename, Exu-Dry®, manufactured by Smith & Nephew.

Example 9 is the antimicrobial layer of Example 8.

### B. Test Procedures

### Moisture Absorption Test:

The amount of moisture absorbed by a substrate over a 24 hour period was determined using the following Moisture Absorption Test:
1. A 20 cm² circle of the substrate was die cut.
2. The cut substrate was weighed (“Initial Weight”).
3. The substrate was placed in a 150 mL container.
4. 100 mL of deionized water was added to the container such that the substrate was completely submerged.
5. The container was allowed to sit uncovered for 24 hours at room temperature.
6. The substrate was removed from the container and suspended in the air with forceps for 1 minute to allow excess fluid to drip from the substrate.
7. The wet sample was weighed (“Final Weight”).

The percent absorbance (% Absorbance) and absorbance per square meter (Absorbance g/m²) of substrate, based on weight, were calculated using routine mathematical calculations.

### Vertical Moisture Wicking Test:

The capillary activity within a substrate will cause water to move vertically up through the substrate. A substrate's ability to vertically wick moisture was determined using the following

### Moisture Wicking Test:

1. A strip of substrate was prepared having a length of 15 cm (in the stitch bonded direction) and a width of 3 cm (in the non-stitch bonded direction).
2. One end of the strip of substrate was pierced horizontally across the 3 cm width with a straight pin. The piercing was made about ½ cm from the edge of the strip. For samples containing a stitch bonded nonwoven material, the stitch bonding was aligned in the vertical direction.
3. Water was added to a flask such that the water level was 14 cm from the top of the flask.
4. Two to three drops of a coloring agent were added to the water.
5. The strip was then suspended in the flask such that the bottom of the strip just comes in contact with the colored water. The straight pin was resting on the mouth of the flask.
6. The strip of substrate was then allowed to wick the colored water for three (3) minutes.
7. After three minutes, the strip of substrate was removed from the flask and the distance traveled by the colored water was measured to the nearest ½ centimeter.

The wicking average and standard deviation values were calculated using routine mathematical calculations.

### Strength Test:

Multi-layered absorptive pads and individual layers comprising the absorptive pads were mechanically tested for tensile strength in the length and width directions. For each sample, the roll (or machine) direction represented the length direction of the sample, while the non-roll (or non-machine) direction represented the width direction of the sample. Stitch bonding was present in the length direction of the samples.

Multi-layer absorptive pads, in whole and in part, were mechanically tested for tensile strength in the length and width directions using a MTS Sintech 10/G mechanical tester with a MTS 25kN load cell.

The strength and modulus of the materials were determined using the following test procedure:

For all samples (except for Example 1), 4″ wide samples were prepared and tested for tensile strength. The samples were held in 3″ wide hydraulic grips with a 10″ span of sample material between the grips.

For Example 1, the sample was tested at its full dimensions (24″×36″) for tensile strength. The sample was held in 3″ wide hydraulic grips with the span of sample material between the grips at approximately 13″.

Since the samples were wider than the grips, one edge of the sample was aligned with the edge of the grips and the other sample edge was free. This was meant to simulate use of the absorptive pad where the pad is not held with uniform pressure. The MTS mechanical testing machine separated the hydraulic grips at a rate of 8 inches/minute, stretching the pad samples. Wetted samples were submerged in phosphate buffered saline (using MPBiomedicals tablets 2810305) for 2 hours and then allowed to drip dry vertically for 5 minutes prior to mechanical testing.

The load that the substrate was able to hold as the strain was increased was calculated as “pounds of force” (“lbf”).

### Antimicrobial Efficacy Test:

Several Gram-positive and Gram-negative bacteria were chosen to illustrate the antimicrobial efficacy of the present absorptive pad. Gram-positive bacteria include, for example and without limitation, Staphylococcus aureus, Staphylococcus epidermidis, and Enterococcus faecalis. Gram-negative bacteria include, for example and without limitation, Klebsiella pneumoniae, Escherichia coli, Acinetobacter baumannii, Enterobacter cloacae, Proteus mirabilis, and Pseudomonas aeruginosa. A selection of these organisms was chosen to demonstrate the antimicrobial efficacy of the Examples herein. However, it should be understood to be within the scope of this invention that similar results would be obtained against other bacteria and/or fungi.

Example 8 was tested for antimicrobial performance. Efficacy against bacteria was assessed with a modified version of AATCC Method 100-1999. Portions (1″×1″) of each sample were placed into a 60 mm Petri dish and inoculated with 4 ml of bacteria suspended in 5% Nutrient Broth in saline. Samples were tested against Staphylococcus aureus ATCC #29213 (“S. aureus”), Klebsiella pneumoniae ATCC #4352 (“K. pneumoniae”), Pseudomonas aeruginosa ATCC #12055 (“P. aeruginosa”), and Acinetobacter baumannii ATCC #19606 (“A. baumannii”). The suspensions of bacteria contained ca. 10E6 cells/ml. After incubation for 24 hours at 37° C., viable cells were recovered by placing each sample in a separate 50 ml centrifuge tube filled with recovery solution (Tryptic Soy Broth supplemented with 0.1% cysteine and 0.01% Tween 80) and vortexing for 30 sec. The number of viable cells in the recovery solution was quantified using a microtiter plate-based “Most-Probable Number” assay. The “Control” sample tested against S. aureus and K. pneumoniae was the non-silver containing product of Example 1. The “Control” sample tested against P. aeruginosa and A. baumannii was the non-silver containing Exu-Dry™ product.

### C. Test Results

### Moisture Absorption Test:

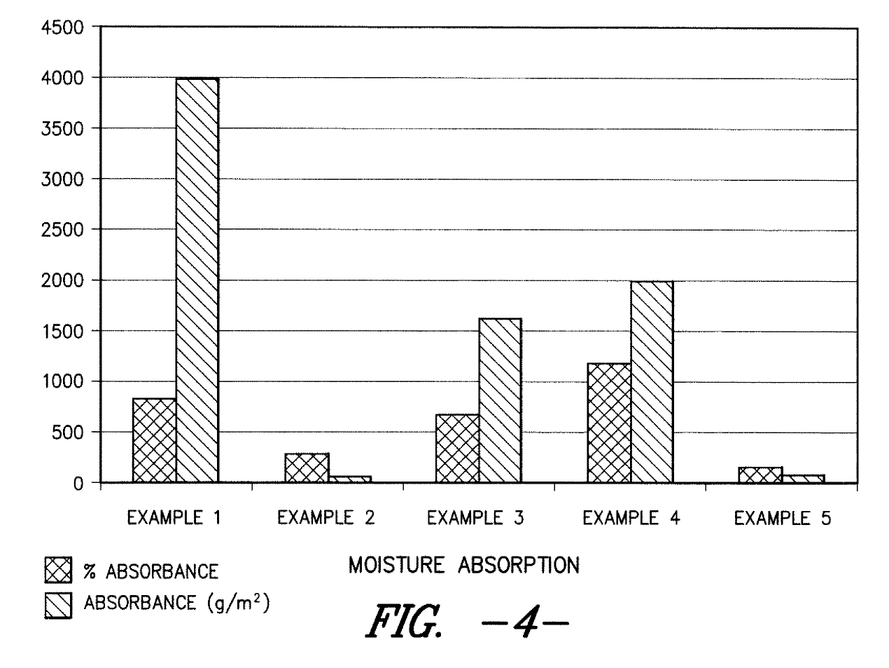

The amount of moisture absorbed by a substrate over a 24 hour period was determined using the Moisture Absorption Test described previously. Test results are provided in Table 1 and FIG. 4. The test results for Examples 8 and 9 and Comparative Example 1 represent an average of triplicate samples that were tested. The test results for the other Examples represent single data points.

TABLE 1
Moisture Absorption Test

| | Initial | Final | | |
|---|---|---|---|---|
| | Weight | Weight | % | Absorbance |
| Sample | (g) | (g) | Absorbance | (g/m²) |
| Example 1 | 0.9732 | 8.9401 | 819 | 3983 |
| (all layers) | | | | |
| Example 2 | 0.0368 | 0.1373 | 273 | 50 |
| (perforated film) | | | | |
| Example 3 | 0.483 | 3.7042 | 667 | 1611 |
| (stitch bonded | | | | |
| nonwoven) | | | | |
| Example 4 | 0.3373 | 4.3154 | 1179 | 1989 |
| (nonwoven, not stitch | | | | |
| bonded) | | | | |
| Example 5 | 0.0909 | 0.2233 | 146 | 66 |
| (occlusive film) | | | | |
| Example 8 | 1.3974 | 10.7589 | 670 | 4769 |
| Example 9 | 0.5462 | 2.2174 | 306 | 851 |
| Comparative Example 1 | 0.6643 | 6.9292 | 943 | 3192 |

### Vertical Moisture Wicking Test:

The ability of a substrate to vertically wick moisture was determined using the Moisture Wicking Test described previously. Test results are provided in Table 2 below.

TABLE 2
Vertical Moisture Wicking Test

| | | | Wicking |
|---|---|---|---|
| | | Wicking | Standard |
| | Wicking | Average | Deviation |
| Sample | (cm) | (cm) | (cm) |
| Example 1 | 9.5 | 9.2 | 0.6 |
| (all layers) | 8.5 | | |
| | 8.5 | | |
| Example 2 | 0.0 | 0.0 | 0.0 |
| (perforated film) | 0.0 | | |
| | 0.0 | | |
| Example 3 | 9.5 | 9.5 | 0.5 |
| (stitch bonded nonwoven) | 10.0 | | |
| | 9.0 | | |
| Example 4 | 4.0 | 4.5 | 0.5 |
| (nonwoven, not stitch | 5.0 | | |
| bonded) | 4.5 | | |
| Example 5 | 0.0 | 0.0 | 0.0 |
| (occlusive film) | 0.0 | | |
| | 0.0 | | |
| Example 8 | 10.0 | 9.5 | 0.9 |
| (all layers with | 8.5 | | |
| antimicrobial layer) | 10.0 | | |
| Example 9 | 9.0 | 8.7 | 0.3 |
| (antimicrobial layer) | 8.5 | | |
| | 8.5 | | |
| Comparative Example 1 | 8.5 | 8.3 | 0.3 |
| | 8.5 | | |
| | 8.0 | | |

### Strength Test:

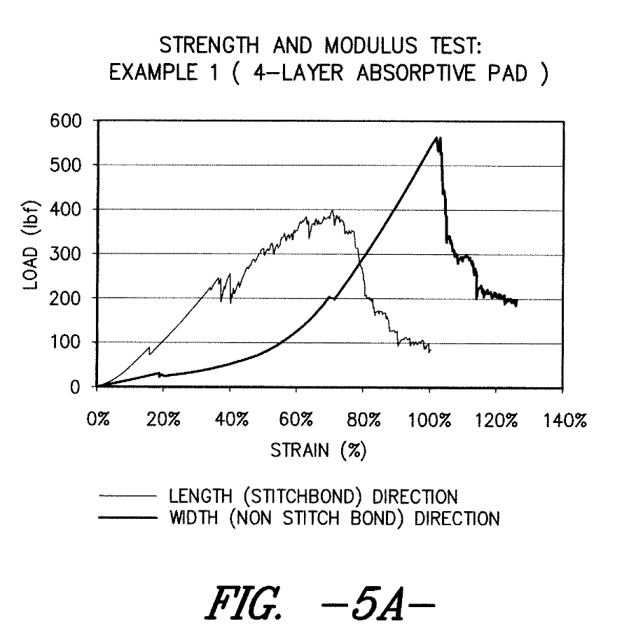

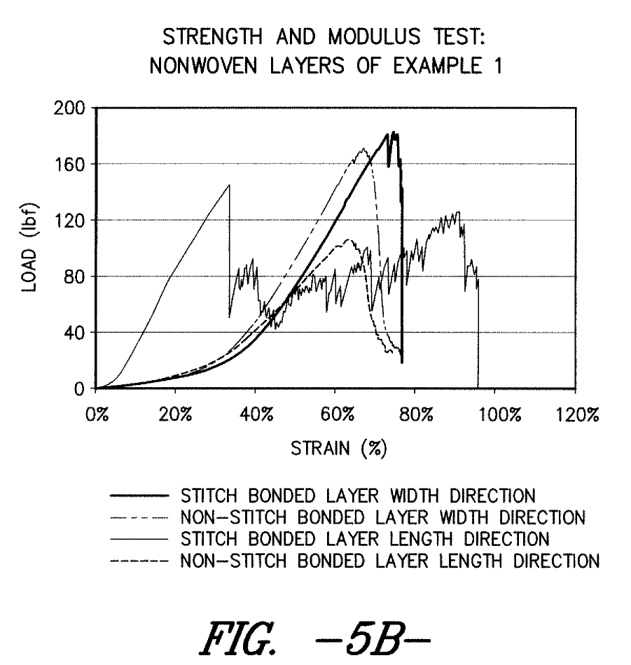

A. Example 1 was tested for mechanical strength in both the length and width directions of the absorptive pad. The test results are shown in Table 3 and FIG. 5A.

As seen in FIG. 5A, the absorptive pad, which includes a stitch bonded layer, exhibited higher tensile strength (i.e. “load”) in the width direction and a higher modulus (i.e. “strain”) in the length direction. FIG. 5A also shows that the load supported by the absorptive pad in the length direction varied above about the 36% strain level. This is believed to be due to the destruction of individual stitch-bonds, as strain and load were increased.

B. Each of the nonwoven layers of Example 1 was tested individually for mechanical strength in both the length and width directions. These test results are provided in Table 3 and are shown in FIG. 5B.

C. Example 6 was the same as Example 1, except that there was no stitch bonding present in either nonwoven layer of the absorptive pad. Example 7 was the same as Example 1, except that both nonwoven layers included stitch bonding. The strength values for each of the nonwoven layers of Examples 6 and 7 were calculated using the results from the nonwoven layers of Example 1. The test results are provided in Table 3.

The test results for the film layers are not provided in Table 3 and are not shown in FIG. 5B because they contributed very little to the overall strength of the absorptive pad, when compared to the nonwoven layers.

TABLE 3
Strength and Modulus Test

| | | Strength | | Modulus | |
|---|---|---|---|---|---|
| | | (Pounds of Force) | | (Pounds of Force/inch) | |
| | | Length | Width | Length | Width |
| Sample | Layer | Direction | Direction | Direction | Direction |
| Example 1 | All 4 layers sewn together | 399 | 563 | 1163 | 1489 |
| Example 1A | All 4 layers stacked together | 155 | 356 | 157 | 217 |
| Individual Layers of | Stitch bonded nonwoven layer | 145 | 181 | 133 | 116 |
| Example 1A | Non-stitch bonded nonwoven | 106 | 171 | 73 | 120 |
| | layer | | | | |
| | Total Composite: | 155 | 356 | 157 | 217 |
| Example 6 | Non-stitch bonded nonwoven | 106 | 171 | 73 | 120 |
| (same as Example | layer | | | | |
| 1, except no stitch | Non-stitch bonded nonwoven | 106 | 171 | 73 | 120 |
| bonding) | layer | | | | |
| | Total Composite: | 212 | 342 | 146 | 240 |
| Example 7 | Stitch bonded nonwoven layer | 145 | 181 | 133 | 116 |
| (same as Example | Stitch bonded nonwoven layer | 145 | 181 | 133 | 116 |
| 1, except both | Total Composite: | 290 | 362 | 266 | 232 |
| nonwoven layers | | | | | |
| are stitch bonded) | | | | | |
| Comparative | Total Composite: | 59 | 37 | 94 | 173 |
| Example 1 | | | | | |
| Wet Comparative | Total Composite: | 51 | 38 | 74 | 139 |
| Example 1 | | | | | |
| Wet Example 1A | Total Composite: | 179 | 386 | 208 | 192 |

Modulus is reported at 50% of ultimate tensile strength. The composite modulus was determined from the tangent of the tension-strain curve of both layers tested together for Example 1. For examples 6 and 7 the total composite modulus is calculated from the sum of the two individual layers. Modulus units are reported as tension (pound of force per unit width of sample) per unit strain (in/in).

Total strength is measured by testing both layers at once in the mechanical tester. The units given are for the entire sample width.

Thus, the stitch bonded absorptive pad exhibits an overall increase in strength and modulus due to the presence of the stitch bonding. The test results illustrate that stitch-bonding increases the strength of the nonwoven layer, and ultimately the multi-layer absorptive pad, in both the length and width direction. However, the strength increase appears to be more significant in the length direction, wherein the increase in strength is about 37%.

In light of these results, it may be desirable to optimize the strength of the absorptive pad by orienting the stitch bonding in the width of the nonwoven absorptive layer, rather than in the length direction. In doing so, a 37% increase in strength from the stitch bonding, combined with the strength of the absorptive pad in the machine direction may result in a stronger overall absorptive pad. Thus, it is possible to align the absorptive material layers, e.g. the nonwoven layers, whether stitch bonded or not, in any configuration needed in order to achieve the desired end-use properties of the absorptive pad.

### Antimicrobial Efficacy:

Example 8 was tested for antimicrobial efficacy by quantitative log reduction against Staphylococcus aureus ATCC #29213 (“S. aureus”), Klebsiella pneumoniae ATCC #4352 (“K. pneumoniae”), Pseudomonas aeruginosa ATCC #12055 (“P. aeruginosa”), and Acinetobacter baumannii ATCC #19606 (“A. baumannii”).

Test results are shown in Table 4. Each value represents an average of two samples (i.e. duplicate samples).

TABLE 4
Antimicrobial Efficacy of Absorptive Pad As Determined By
Quantitative Log Reduction

| | | | | Log |
|---|---|---|---|---|
| | | | | Reduction |
| | | | | After 24 |
| | | | | Hours: |
| | | Control | | Example |
| | Initial | Log After 24 | Example 8 | 8 vs. |
| | Bioburden | Hours | Log After 24 | Control |
| | (# cells/ | (# cells/ | Hours | (# cells/ |
| Microbe | sample) | sample) | (# cells/sample) | sample) |
| S. aureus | 6.41 | 7.01 | 3.62 | 3.39 |
| ATCC #29213 | | | | |
| K. pneumoniae | 6.14 | 7.17 | 2.78 | 4.39 |
| ATCC #4352 | | | | |
| P. aeruginosa | 4.93 | 7.92 | 1.15 | 6.77 |
| ATCC #12055 | | | | |
| A. baumannii | 4.69 | 7.92 | 2.45 | 5.47 |
| ATCC #19606 | | | | |

The results show that Example 8 exhibits good antimicrobial efficacy against both Gram positive and Gram negative microbes.

These and other modifications and variations to the present invention may be practiced by those of ordinary skill in the art, without departing from the spirit and scope of the present invention. Furthermore, those of ordinary skill in the art will appreciate that the foregoing description is by way of example only, and is not intended to limit the scope of the invention described in the appended claims.

We claim:

1. An absorptive pad comprising the layers of:
(a) at least one skin-contact layer of apertured hydrophobic film;
(b) optionally, at least one layer of non-electrically conductive absorptive material that contains an antimicrobial agent;
(c) at least one absorptive layer that contains at least one reinforcement material;
(d) optionally, at least one absorptive reservoir layer; and
(e) at least one layer of occlusive film comprising the outermost layer of the absorptive pad;
wherein each of layers (a)-(e) are arranged substantially coextensive with each other.

2. The absorptive pad of claim 1, wherein layer “b” is a fabric.

3. The absorptive pad of claim 2, wherein the fabric is comprised of a blend of nylon and polyester fiber.

4. The absorptive pad of claim 3, wherein the fabric is comprised of a blend of nylon, polyester, and spandex fiber.

5. The absorptive pad of claim 1, wherein the antimicrobial agent of layer “b” is a silver-ion containing compound.

6. The absorptive pad of claim 5, wherein the silver-ion containing compound is a silver zirconium phosphate compound.

7. The absorptive pad of claim 5, wherein layer “b” further contains a binding agent.

8. The absorptive pad of claim 7, wherein the binding agent is a polyurethane-based compound.

9. The absorptive pad of claim 1, wherein layer “c” is a fabric.

10. The absorptive pad of claim 9, wherein the fabric is comprised of a blend of nylon and polyester fiber.

11. The absorptive pad of claim 9, wherein the at least one reinforcement material is yarn stitch bonded throughout the fabric.

12. The absorptive pad of claim 9, wherein the at least one reinforcement material is long staple fiber.

13. The absorptive pad of claim 1, wherein layer “d” is a fabric.

14. The absorptive pad of claim 13, wherein the fabric is comprised of a blend of nylon and polyester fiber.

15. The absorptive pad of claim 1, wherein layers “c” and “d” are fabrics comprised of 100% synthetic fibers.

16. The absorptive pad of claim 1, wherein the apertured hydrophobic film layer is comprised of polyethylene.

17. The absorptive pad of claim 1, wherein the at least one layer of occlusive film is comprised of polyethylene.

18. The absorptive pad of claim 1, wherein at least one of layers (b), (c), and (d) contain an antimicrobial agent.

19. The absorptive pad of claim 1, wherein the layers comprising the absorptive pad are sewn together.

20. An absorptive pad comprising the sequential layers of:
(a) at least one skin-contact layer of apertured hydrophobic film;
(b) optionally, at least one layer of non-electrically conductive absorptive material that contains an antimicrobial agent;
(c) at least one absorptive layer that contains at least one reinforcement material;
(d) at least one absorptive reservoir layer; and
(e) at least one layer of occlusive film;
wherein each of layers (a)-(e) are arranged substantially coextensive with each other.

21. An absorptive pad comprising the sequential layers of:
(a) at least one skin-contact layer of apertured hydrophobic film;
(b) at least one layer of non-electrically conductive absorptive material that contains an antimicrobial agent;
(c) at least one absorptive layer that contains at least one reinforcement material;
(d) at least one absorptive reservoir layer;
(e) at least one layer of occlusive film;
wherein each of layers (a)-(e) are arranged substantially coextensive with each other.
